(19) United States
(12) Patent Application Publication
(10) Pub. No.: US 2025/0375749 A1
(43) Pub. Date: Dec. 11, 2025

(54) CORE-SHELL NANOPARTICLES HAVING GOLD NANOSHELL, AND METHOD FOR MANUFACTURING SAID CORE-SHELL NANOPARTICLES

(71) Applicant: TANAKA PRECIOUS METAL TECHNOLOGIES Co., Ltd., Tokyo (JP)

(72) Inventor: Hiroshi KISHI, Tokyo (JP)

(73) Assignee: TANAKA PRECIOUS METAL TECHNOLOGIES Co., Ltd., Tokyo (JP)

(21) Appl. No.: 18/684,379

(22) PCT Filed: Aug. 26, 2022
(86) PCT No.: PCT/JP2022/032179

§ 371 Date: May 21, 2024

(30) Foreign Application Priority Data
Aug. 26, 2021 (JP) 2021-138003

(51) Int. Cl. B01J 13/12; B82Y 30/00; B82Y 40/00

(52) U.S. Cl. CPC B01J 13/12; B82Y 30/00; B82Y 40/00

(57) ABSTRACT

Provided are core-shell nanoparticles having a gold nanoshell and having high dispersion stability, and a method for manufacturing the core-shell nanoparticles. A method for manufacturing core-shell particles having, on the core particle surface, a gold nanoshell and a protective agent, the manufacturing method including: (a) a step for mixing a solution of core particles and a solution of gold nanoclusters; (b) a step for adding a protective agent and a reducing agent, stirring the components, and adding a gold complex to form a gold nanoshell on the surface of the core particles; and (c) a step for recovering the core-shell particles produced in step (b).

### TECHNICAL FIELD

The present invention relates to core-shell nanoparticles having gold nanoshells and a method for producing the same.

### BACKGROUND ART

Core-shell nanoparticles having gold nanoshells are known as a coloring agent exhibiting surface plasmon resonance at a predetermined wavelength, particularly from red light to near infrared light, and Patent Document 1 discloses core-shell nanoparticles having gold nanoshells as an optical material. In addition, Non-Patent Document 1 discloses core-shell nanoparticles having gold nanoshells as biomarkers.

However, the conventional method for producing core-shell nanoparticles having gold nanoshells has such a problem that the particle concentration in the reaction system at the time of production is significantly dilute as disclosed in Non-Patent Document 1. The reason why the production method at a high particle concentration is important in the production of the core-shell nanoparticles is that as the core-shell nanoparticles are produced at a higher concentration, the number of core-shell nanoparticles obtained per lot increases, resulting in advantages in terms of cost and environmental load.

In addition, in the conventional method for producing core-shell nanoparticles having gold nanoshells, protective agents for dispersing and stabilizing the core-shell nanoparticles were insufficiently considered, and therefore conventional methods has not been optimized to obtain core-shell nanoparticles exhibiting sufficient dispersion stability and optical properties.

### CITATION LIST

### Patent Document

Patent Document 1: JP 2016-212268 A

### Non-Patent Document

Non-Patent Document 1: Chem. Sci., 2017, 8, 3038

### SUMMARY OF THE INVENTION

### Problems to be Solved by the Invention

Conventional methods for producing core-shell nanoparticles having gold nanoshells are designed as a reaction system using a dilute particle concentration, that is, using a dilute concentration of gold ion which is a raw material. An object of the present invention is to provide a method for producing core-shell nanoparticles having gold nanoshells by a reaction system using a higher concentration of gold ions as a raw material compared with conventional methods, and core-shell nanoparticles having gold nanoshells produced according to the production method, with high dispersion stability.

### Means for Solving the Problems

In order to solve the above problems, the inventors of the present invention have sought a reducing agent for reducing gold ions which is as a raw material of core-shell nanoparticles having gold nanoshells, and further have intensively studied a protective agent for dispersing and stabilizing core-shell nanoparticles obtained when a selected reducing agent is used, thereby achieving the present invention.

That is, the present invention provides core-shell nanoparticles having gold nanoshells having a protective agent, which have high dispersion stability in an aqueous solution or in a polar solvent optionally miscible with water, and have gold nanoshells exhibiting surface plasmon resonance from red light to near infrared light, and further provides a method for producing the core-shell nanoparticles.

### Effects of the Invention

In one aspect of the present invention, the method for producing core-shell nanoparticles having gold nanoshells uses a reducing agent in the production step, wherein the reducing agent is a compound represented by the following chemical formula (1):

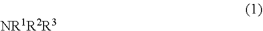

wherein R¹ is a C¹ to C⁴ hydroxyalkyl group, R² is a C¹ to C⁴ carboxyalkyl group, and R³ is a C¹ to C⁴ hydroxyalkyl group, or a C¹ to C⁴ carboxyalkyl group. In another aspect, in the core-shell nanoparticles having gold nanoshells of the present invention, a protective agent for dispersing and stabilizing the core-shell nanoparticles having gold nanoshells produced when the reducing agent is used is partially saponified polyvinyl alcohol having an average polymerization degree of about 500.

The core-shell nanoparticles having gold nanoshells produced by the method of the present invention have excellent dispersion stability in water and in polar solvents optionally miscible with water, and exhibit surface plasmon resonance from red light to near-infrared light.

### BRIEF DESCRIPTION OF DRAWINGS

FIG. 1 shows each absorbance spectrum of the core-shell nanoparticles according to the present invention and the particles of Comparative Examples.

FIG. 2 shows TEM images of the core-shell nanoparticles according to the present invention and the particles of Comparative Examples.

FIG. 3 shows each absorbance spectrum of the core-shell nanoparticles according to the present invention and the particles of Comparative Examples.

FIG. 4 shows each absorbance spectrum of the core-shell nanoparticles of the present invention and the particles of Comparative Examples.

### DESCRIPTION OF EMBODIMENTS

In one embodiment, the present invention is as follows.

### 1

A method for producing core-shell particles having gold nanoshells and a protective agent on surfaces of core particles, comprising the following steps:
(a) mixing a solution of core particles and a solution of gold nanoclusters;
(b) adding a protective agent and a reducing agent and stirring, and further adding a gold complex to form gold nanoshells on surfaces of the core particles; and
(c) recovering the core-shell particles produced in the step (b), wherein
the reducing agent is a compound represented by Chemical Formula (1):

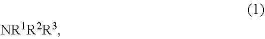

wherein R¹ is a C to C₄ hydroxyalkyl group, R² is a C₁ to C₄ carboxyalkyl group, and R³ is a C₁ to C₄ hydroxyalkyl group, or a C₁ to C₄ carboxyalkyl group.

### 2

The method according to [1], wherein the reducing agent is bicine or N, N-bis (carboxymethyl) ethanolamine.

### 3

The method according to [1], wherein the reducing agent is bicine.

### 4

The method according to [1], wherein the protective agent is partially saponified polyvinyl alcohol having an average polymerization degree of 300 to 700.

### 5

The method according to [1], wherein the protective agent is modified partially saponified polyvinyl alcohol with an average polymerization degree of 300 to 700, having sulfonic acid group introduced.

### 6

The method according to [1], wherein in the step (b), the formation of gold nanoshells on surfaces of core particles is performed in the presence of a gold ion at a concentration of 5 mM or more.

### 7

The method according to [1], wherein in the step (b), the formation of gold nanoshells on surfaces of core particles is performed within 10 minutes at room temperature.

### 8

The method according to [1], wherein the core particles have a diameter of 50 nm to 300 nm.

### 9

The method according to [1], wherein the gold nanoshells have a thickness of 15 nm or less.

Core-shell nanoparticles comprising gold nanoshells and a protective agent on surfaces of core particles, wherein the gold nanoshells have a thickness of 15 nm or less, wherein the core particles have a diameter of 50 nm to 300 nm, and wherein the protective agent is partially saponified polyvinyl alcohol having an average polymerization degree of 300 to 700.

The term “gold nanoshell(s)” herein refers to gold shell(s) to be formed on the surfaces of core particles, which have a thickness of 15 nm or less.

The term “core-shell nanoparticle(s)” herein refers to particle(s) having a diameter of 1 um or less, in which shells are formed on the surfaces of core particles. The shells are formed using a different material from the core particles.

The term “reducing agent” herein refers to an organic compound used to reduce gold complexes to deposit gold nanoshells.

The term “protective agent” herein refers to a water-soluble polymer compound for retaining dispersion stability of nanoparticles in an aqueous solution by being adsorbed on the surfaces of core-shell nanoparticles having gold nanoshells.

Examples of the water-soluble polymer compound include, but are not limited to, polyvinyl alcohol, dextran, inulin, polyvinyl pyrrolidone, polyacrylic acid, and polyoxazoline. Such polymer compound as an example herein may include a compound with a substituent. Examples of the substituent of the polymer compound include a carbonyl group and a sulfonic acid group.

The polymerization degree of the protective agent can be measured by a known method. As an example, the polymerization degree of polyvinyl alcohol can be measured by a solution viscosity measurement method in accordance with JIS K6726-1994.

The term “core particles” refers to template particles for forming gold nanoshells, which are made of an inorganic material or an organic polymer. Examples of the inorganic material or the organic polymer include, but are not limited to, silicon dioxide, titanium dioxide, zinc sulfide, silver, copper, and polystyrene.

The term “diameter of core particles” herein refers to the average of a predetermined number of core particle diameters observed by a transmission electron microscope (TEM).

The term “seed particle” herein refers to a particle serving as a starting point for forming a nanoshell on the surface of a core particle, and the particle is about 2 nm in diameter. A seed particle is preferably a gold nanocluster having a diameter of about 2 nm.

One embodiment according to the present invention is a method for producing core-shell nanoparticles having gold nanoshells. The method comprises the following steps (a) to (c) in this order:
(a) mixing a solution of core particles and a solution of gold nanoclusters;
(b) adding a protective agent and a reducing agent and stirring, and further adding a gold complex to form gold nanoshells on the surfaces of the core particles; and
(c) recovering the core-shell nanoparticles produced in the step (b).

The “reducing agent” in the step (b) is preferably a compound represented by a formula (1):

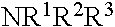

wherein R¹ is a C₁ to C₄ hydroxyalkyl group, R² is a C₁ to C₄ carboxyalkyl group, and R³ is a C to C₄ hydroxyalkyl group, or a C₁ to C₄ carboxyalkyl group.

The reducing agent is more preferably bicine or N,N-bis(carboxymethyl)ethanolamine, and further preferably bicine.

The amount of the reducing agent to be added in the step (b) is preferably 6.7 to 40 times the molar ratio of gold, and more preferably 10 to 25 times.

The pH of the reducing agent in the step (b) is preferably 7.7 to 8.6 and more preferably 7.7 to 8.3.

The “protective agent” in the step (b) is preferably polyvinyl alcohol, dextran, inulin, polyvinylpyrrolidone, polyacrylic acid, or polyoxazoline, and more preferably polyvinyl alcohol.

The polyvinyl alcohol as a protective agent is preferably a partially saponified type. The polyvinyl alcohol has further the average polymerization degree of preferably 1000 or less, more preferably 300 to 700, and still more preferably 500.

In one embodiment, the polyvinyl alcohol in the present invention may be ASP-05 (partially saponified polyvinyl alcohol having an average polymerization degree of 500 and further modified by a sulfonic acid group) manufactured by JAPAN VAM & POVAL CO., LTD.

The total weight percent of the protective agent in the solution upon the completion of the step (b) (hereinafter referred to as the final concentration of the protective agent) is preferably 0.01% to 2%, and more preferably 0.4% to 1%.

The “gold complex” in the step (b) is preferably chloroauric acid, a cyanoaurate, or gold sulfite, and more preferably chloroauric acid.

The gold complex concentration in the step (b) is preferably 2 mM to 14 mM, and more preferably 2 mM to 10 mM.

One embodiment according to the present invention relates to core-shell nanoparticles having gold nanoshells coated with a protective agent, produced using bicine as a reducing agent. The protective agent is polyvinyl alcohol.

The protective agent is preferably a partially saponified polyvinyl alcohol, and more preferably a sulfonation-modified polyvinyl alcohol having the above-described properties.

Another embodiment according to the present invention relates to a dispersion in which core-shell nanoparticles having gold nanoshells are dispersed in an aqueous solution.

### Estimation of Shell Thickness

The term “shell thickness” is defined herein as a value estimated according to the following way.

The core-shell nanoparticle having gold nanoshells according to the present invention exhibits surface plasmon resonance in red light to near infrared light region in the state where the core-shell nanoparticles are dispersed in aqueous solution.

The measurement of an absorbance spectrum from visible light to near infrared light due to the plasmon resonance in the core-shell nanoparticle dispersion can provide quantification of the shell thickness of the core-shell nanoparticles according to the present invention. Therefore, the formation of core-shell nanoparticles having a desired shell thickness can be predicted by, for instance, comparing a measured value of the maximum absorption wavelength of the plasmon resonance with the calculated value.

As an example, when 55 nm-diameter SiO₂ as core particles is used for forming the core-shell nanoparticles, the calculated value of the maximum absorption wavelength corresponding 9 nm shell thickness is 644 nm, corresponding to 10 nm shell thickness is 632 nm, and corresponding to 11 nm shell thickness is 624 nm. Therefore, if as a result of measuring absorbance spectrum, the measured value of the maximum absorption wavelength of the core-shell nanoparticle dispersion is between 624 nm and 644 nm, it is predicted that the gold nanoshell thickness is about 9 nm to 11 nm.

As a further example, when 80 nm-diameter SiO₂ as core particles is used for forming the core-shell nanoparticles, the calculated value of the maximum absorption wavelength corresponding 9 nm shell thickness is 724 nm, corresponding to 10 nm shell thickness is 706 nm, and corresponding to 11 nm shell thickness is 694 nm. Therefore, if as a result of measuring the absorbance spectrum the measured value of the maximum absorption wavelength of the core-shell nanoparticle dispersion is between 694 nm and 724 nm, it is predicted that the gold nanoshell thickness is about 9 nm to 11 nm.

As a further example, when 200 nm-diameter SiO₂ as the core particles is used for forming the core-shell nanoparticles, the calculated value of the maximum absorption wavelength corresponding 9 nm shell thickness is 790 nm, corresponding to 10 nm shell thickness is 772 nm, and corresponding to 11 nm shell thickness is 754 nm. Therefore, if as a result of measuring the absorbance spectrum the measured value of the maximum absorption wavelength of the core-shell nanoparticle dispersion is between 754 nm and 790 nm, it is predicted that the gold nanoshell thickness is about 9 nm to 11.

The core-shell nanoparticles having gold nanoshells according to the present invention can be widely industrially applied for, for instance, a color sensor and a biomarker using plasmon resonance, a photoelectric conversion material and a photothermal conversion material with optical properties for absorbing red light to near infrared light, a near infrared light condensing material for a photocatalyst in a water decomposition reaction, and a supporting body for photo upconversion.

Numbers expressing quantities in the specification and in the claims relating to, for instance, components or attributes may be interpreted as modified by the modifier “about”. The term “about” means that a margin of error or variation may be included without changing the essence of the present invention, and that a variation of ±10% of a numerical value is allowed unless otherwise specified.

### EXAMPLES

Hereinafter, the present invention will be described more specifically with reference to Examples. However, the scope of the present invention is not limited to the aspects shown in the following Examples.

Temperatures indicated in Examples and Comparative Examples herein refer to temperatures in the liquid state unless otherwise specified. In addition, unless otherwise specified, “%” refers to weight %.

In Examples, chemical agents manufactured by the following companies were used unless otherwise specified.
Ethanol (manufactured by FUJIFILM Wako Pure Chemical Corporation)
Tetraethyl orthosilicate (manufactured by Tokyo Chemical Industry Co., Ltd.)
Ammonia water (manufactured by FUJIFILM Wako Pure Chemical Corporation)
Hydrochloric acid (manufactured by FUJIFILM Wako Pure Chemical Corporation)
3-aminopropyltrimethoxysilane (manufactured by Tokyo Chemical Industry Co., Ltd.)
Chloroauric acid aqueous solution (manufactured by Tanaka Kikinzoku Kogyo K.K.)
Sodium borohydride (manufactured by FUJIFILM Wako Pure Chemical Corporation)
Bicine (manufactured by DOJINDO LABORATORIES)
The following devices were used in Example below, unless otherwise specified.
Rotary evaporator (N-1300, manufactured by Tokyo Rika Machine Co., Ltd.)
Dryer (DV600, manufactured by Yamato Scientific Co., Ltd.)
Centrifuge (Model 3500, manufactured by Kubota Shoji Co., Ltd.)

### Example 1

(Synthesis of SiO₂ Core Particles) 150 mL of ethanol and 7.5 mL of tetraethyl orthosilicate were mixed, and then the mixture was stirred at 50° C. for 30 minutes. While the solution was stirred at 50° C., 30 mL of 3% by weight of ammonia water was added thereto, and the mixture was stirred at 50° C. for 2 hours to provide a crude SiO₂ particle dispersion.

50 mL of ultrapure water was added to the SiO₂ particle dispersion, and the mixture was concentrated in vacuo by a rotary evaporator to remove ammonia and ethanol. The concentrated solution was dialyzed overnight in deionized water to provide a SiO₂ particle dispersion having about 55 nm-diameter particles.

### (Introduction of Amino Group to Surface of SiO₂ Core Particles)

To a mixed solution of 7 mL of ethanol, 2 mL of 1 M hydrochloric acid, and 0.35 mL of 3-aminopropyltrimethoxysilane, the SiO₂ particle dispersion containing 0.1 mmol of hydrochloric acid and 0.2 g of SiO₂ particles was added while stirring the mixture at 25° C.

The mixed solution was stirred for 4 hours in a dryer set at 70° C. to cause a reaction.

After completion of the reaction, the reaction mixture was left at room temperature to dissipate heat.

The reaction mixture was centrifuged at a centrifugal acceleration of 15,000 G for 5 minutes in a centrifuge, and then precipitates were recovered. The precipitates were redispersed in 1 mL of 10 mM acetic acid. The washing operation was performed 3 times. Finally, amino group-introduced SiO₂ particles were obtained in a dispersion state in 10 mM acetic acid.

### (Synthesis of Seed Particles)

1.78 mL of ultrapure water, 0.55 mL of 5% by weight of polyvinyl alcohol (manufactured by Sigma-Aldrich Japan, molecular weight: 10,000), and 0.17 mL of a 15 mM chloroauric acid aqueous solution were mixed, and stirred at 4° C. for 10 minutes.

0.25 mL of 0.1 M sodium borohydride was added to the mixed solution, and further the mixture was continuously stirred in an ice bath at 4° C. for 90 minutes.

The reaction mixture was dialyzed with deionized water overnight to provide a seed particle dispersion.

(Formation of Gold Nanoshells on Surface of Core Particles) 500 μL of a seed particle dispersion and 70 μL of an amino group-introduced SiO₂ particle dispersion adjusted to 1% by weight were mixed in a 1.5 mL microtube, and the mixture was subjected to ultrasonic irradiation for 30 seconds.

After the ultrasonic dispersion treatment, 100 μL of the solution was added to a 6 mL glass vial. To the glass vial, 100 μL of deionized water, 200 μL of 5% by weight of polyvinyl alcohol (JP-10 manufactured by JAPAN VAM & POVAL CO., LTD., average polymerization degree 1000, partially saponified type), and 250 μL of 0.6 M bicine (reducing agent 1, manufactured by DOJINDO LABORATORIES) adjusted to pH 8 were added while stirring with a magnetic stirrer.

To the solution, 0.5 mL of a 15 mM chloroauric acid aqueous solution was added, and then the mixed solution was continuously stirred at 25° C. for 10 minutes.

The reaction mixture was centrifuged at a centrifugal acceleration of 4,000 G for 5 minutes with a centrifuge to recover a precipitate, and a washing operation of redispersing the precipitate in 0.5 mL of deionized water was repeated 3 times. Finally, the mixture was dispersed in 0.5 mL of deionized water to provide a dispersion of core-shell nanoparticles having gold nanoshells.

The absorbance of the obtained core-shell nanoparticle dispersion was measured.

### Example 2

A dispersion of core-shell nanoparticles having gold nanoshells was obtained in the same manner as in Example 1 except that in the above process of gold nanoshell formation on surface of core particles, bicine as Reducing agent 1 was replaced with N,N-bis(carboxymethyl)ethanolamine as Reducing agent 2.

### Comparative Example 1

A dispersion of core-shell nanoparticles having gold nanoshells was obtained in the same manner as in Example 1 except that in the above process of gold nanoshell formation on surface of core particles, bicine as Reducing agent 1 was replaced with 2-hydroxy-3-morpholinopropanesulfonic acid (manufactured by DOJINDO LABORATORIES) as Reducing agent 3.

### Comparative Example 2

A dispersion of core-shell nanoparticles having gold nanoshells was obtained in the same manner as in Example 1 except that in the above process of gold nanoshell formation on surface of core particles, bicine as Reducing agent 1 was replaced with bis(2-hydroxyethyl)aminotris(hydroxymethyl)methane (manufactured by DOJINDO LABORATORIES) as Reducing agent 4.

### Comparative Example 3

A dispersion of core-shell nanoparticles having gold nanoshells was obtained in the same manner as in Example 1 except that in the above process of gold nanoshells formation on surface of core particles, bicine as Reducing agent 1 was replaced with 1, 3-diamino-2-propanol-N,N, N′,N′-tetraacetic acid (manufactured by Tokyo Chemical Industry Co., Ltd) as Reducing agent 5.

### Comparative Example 4

A dispersion of core-shell nanoparticles having gold nanoshells was obtained in the same manner as in Example 1 except that in the above process of gold nanoshell formation on surface of core particles, bicine as Reducing agent 1 was replaced with N-(2-hydroxyethyl) ethylenediamine-N,N′,N′-triacetic acid (manufactured by Tokyo Chemical Industry Co., Ltd)) as Reducing agent 6.

### Comparative Example 5

A dispersion of core-shell nanoparticles having gold nanoshells was obtained in the same manner as in Example 1 except that in the above process of gold nanoshell formation on surface of core particles, bicine as Reducing agent 1 was replaced with o 1, 3-bis[tris (hydroxymethyl)methylamino]propane (manufactured by Tokyo Chemical Industry Co., Ltd) as Reducing agent 7.

### Comparative Example 6

A dispersion of core-shell nanoparticles having gold nanoshells was obtained in the same manner as in Example 1 except that in the above process of gold nanoshell formation on surface of core particles, bicine as Reducing agent 1 was replaced with N,N-bis (2-hydroxyethyl)-2-aminoethanesulfonic acid (manufactured by DOJINDO LABORATORIES) as Reducing agent 8.

### Comparative Example 7

A dispersion of core-shell nanoparticles having gold nanoshells was obtained in the same manner as in Example 1 except that in the above process of gold nanoshell formation on surface of core particles bicine as Reducing agent 1 was replaced with N-(2-acetamide)iminodiacetic acid (manufactured by DOJINDO LABORATORIES) as Reducing agent 9.

### Comparative Example 8

A dispersion of core-shell nanoparticles having gold nanoshells was obtained in the same manner as in Example 1 except that in the above process of gold nanoshell formation on surface of core particles bicine as Reducing agent 1 was replaced with 4-(2-hydroxyethyl)-1-piperazinepropanesulfonic acid (manufactured by DOJINDO LABORATORIES) as Reducing agent 10.

### Comparative Example 9

A dispersion of core-shell nanoparticles having gold nanoshells was obtained in the same manner as in Example 1except that in the above process of gold nanoshell formation on surface of core particles, bicine as Reducing agent 1 was replaced with 4-(2-hydroxyethyl) piperazine-1-ylethanesulfonic acid (manufactured by DOJINDO LABORATORIES) as Reducing agent 11.

### Comparative Example 10

A dispersion of core-shell nanoparticles having gold nanoshells was obtained in the same manner as in Example 1 except that in the above process of gold nanoshell formation on surface of core particles, bicine as Reducing agent 1 was replaced with 4-(2-hydroxyethyl)piperazine-1-(2-hydroxypropane-3-sulfonic acid) hydrate (manufactured by DOJINDO LABORATORIES) as Reducing agent 12.

### Comparative Example 11

A dispersion of core-shell nanoparticles having gold nanoshells was obtained in the same manner as in Example 1 except that in the above process of gold nanoshell formation on surface of core particles, bicine as Reducing agent 1 was replaced with N-tri(hydroxymethyl)methylglycine (manufactured by DOJINDO LABORATORIES) as Reducing agent 13.

### Comparative Example 12

A dispersion of core-shell nanoparticles having gold nanoshells was obtained in the same manner as in Example 1 except that in the above process of gold nanoshell formation on surface of core particles, bicine as Reducing agent 1 was replaced with tris (hydroxymethyl) aminomethane (FUJIFILM Wako Pure Chemical Corporation) as Reducing agent 14.

### Comparative Example 13

A dispersion of core-shell nanoparticles having gold nanoshells was obtained in the same manner as in Example 1 except that in the above process of gold nanoshell formation on surface of core particles, bicine as Reducing agent 1 was replaced with L-serine (manufactured by Tokyo Chemical Industry Co., Ltd) as Reducing agent 15.

### Comparative Example 14

A dispersion of core-shell nanoparticles having gold nanoshells was obtained in the same manner as in Example 1 except that in the above process of gold nanoshell formation on surface of core particles, bicine as Reducing agent 1 was replaced with sodium L (+)-ascorbate (FUJIFILM Wako Pure Chemical Corporation) as Reducing agent 16.

### Example 3

(Synthesis of SiO₂ core particles)

A 80 nm-diameter SiO₂ particle dispersion was obtained in the same manner as in Example 1 except that the reaction temperature was 45° C.

### (Introduction of Amino Group to Surface of SiO₂ Core Particles)

An amino group-introduced SiO₂ particle dispersion was obtained in the same manner as in Example 1 except that the 80 nm-diameter SiO₂ particle dispersion was used.

### (Synthesis of Seed Particles)

The same procedure as in Example 1 was performed to provide a seed particle dispersion.

### (Formation of Gold Nanoshell on Surface of Core Particles)

500 μL of a seed particle dispersion and 100 μL of an amino group-introduced Si2 particle dispersion adjusted to 1% by weight were mixed in a 1.5 mL microtube, and the mixture was subjected to ultrasonic irradiation for 30 seconds.

After the ultrasonic dispersion treatment, 100 μL of the solution was added to a 6 mL glass vial. To the glass vial, 390 μL of deionized water, 10 μL of 5% by weight of polyvinyl alcohol (DM-17 manufactured by JAPAN VAM & POVAL CO., LTD., partially saponified type having an average polymerization degree of 1700, carbonyl group-introduced modified polyvinyl alcohol), and 150 μL of 1 M bicine adjusted to pH 8 were added therein while stirring with a magnetic stirrer.

To the solution, 0.5 mL of a 15 mM chloroauric acid aqueous solution was added, and then the mixed solution was continuously stirred at 25° C. for 1 hour.

The reaction mixture was centrifuged at a centrifugal acceleration of 4,000 G for 5 minutes with a centrifuge to recover a precipitate, and a washing operation of redispersing the precipitate in 0.5 mL of deionized water was repeated 3 times. Finally, the mixture was dispersed in 0.5 mL of deionized water to provide a dispersion of core-shell nanoparticles having gold nanoshells.

The absorbance of the obtained core-shell nanoparticle dispersion was measured.

### Example 4

A dispersion of core-shell nanoparticles having gold nanoshells was obtained in the same manner as in Example 3 except that in the above process of gold nanoshell formation on surface of core particles, 300 μL of deionized water and 100 μL of 5% by weight of polyvinyl alcohol (DM-17 manufactured by JAPAN VAM & POVAL CO., LTD., partially saponified type having an average polymerization degree of 1700, carbonyl group-introduced modified polyvinyl alcohol) were used.

### Example 5

A dispersion of core-shell nanoparticles having gold nanoshells was obtained in the same manner as in Example 3 except that in the above process of gold nanoshells formation on surface of core particles, 200 μL of deionized water and 200 μL of 5% by weight of polyvinyl alcohol (DM-17 manufactured by JAPAN VAM & POVAL CO., LTD., partially saponified type having an average polymerization degree of 1700, carbonyl group-introduced modified polyvinyl alcohol) were used.

### Example 6

A dispersion of core-shell nanoparticles having gold nanoshells was obtained in the same manner as in Example 3 except that in the above process of gold nanoshell formation on surface of core particles, 50 μL of deionized water and 350 μL of 5% by weight of polyvinyl alcohol (DM-17 manufactured by JAPAN VAM & POVAL CO., LTD., partially saponified type having an average polymerization degree of 1700, carbonyl group-introduced modified polyvinyl alcohol) were used.

### Example 7

A dispersion of core-shell nanoparticles having gold nanoshells was obtained in the same manner as in Example 5, except that in the above process of gold nanoshell formation on surface of core particles, the protective agent was changed into 5% by weight of polyvinyl alcohol (ASP-05 manufactured by JAPAN VAM & POVAL CO., LTD., partially saponified type having an average polymerization degree of 500, sulfonic acid group-introduced modified polyvinyl alcohol), and further that 225 μL of the deionized water and 125 μL of 1 M bicine adjusted to pH 8 were used.

### Example 8

A dispersion of core-shell nanoparticles having gold nanoshells was obtained in the same manner as in Example 7 except that in the above process of gold nanoshell formation on surface of core particles, 200 μL of deionized water and 150 μL of 1 M bicine adjusted to pH 8 were used.

### Example 9

A dispersion of core-shell nanoparticles having gold nanoshells was obtained in the same manner as in Example 7 except that in the above process of gold nanoshell formation on surface of core particles, 150 μL of deionized water and 200 μL of 1 M bicine adjusted to pH 8 were used.

### Example 10

A dispersion of core-shell nanoparticles having gold nanoshells was obtained in the same manner as in Example 7 except that in the above process of gold nanoshell formation on surface of core particles, no deionized water and 350 μL of 1 M bicine adjusted to pH 8 were used.

### Example 11

A dispersion of core-shell nanoparticles having gold nanoshells was obtained in the same manner as in Example 8 except that in the above process of gold nanoshell formation on surface of core particles, the pH of 1 M bicine was changed to pH 7.7.

### Example 12

A dispersion of core-shell nanoparticles having gold nanoshells was obtained in the same manner as in Example 8 except that in the above process of gold nanoshells formation on surface of core particles, the pH of 1 M bicine was changed to pH 8.3.

### Example 13

A dispersion of core-shell nanoparticles having gold nanoshells was obtained in the same manner as in Example 8 except that in the above process of gold nanoshell formation on surface of core particles, the pH of 1 M bicine was changed to pH 8.6.

### Example 14

A dispersion of core-shell nanoparticles having gold nanoshells was obtained in the same manner as in Example 8 except that in the above process of gold nanoshell formation on surface of core particles, 5 mM chloroauric acid aqueous solution was used.

### Example 15

A dispersion of core-shell nanoparticles having gold nanoshells was obtained in the same manner as in Example 8 except that in the above process of gold nanoshell formation on surface of core particles, 10 μL of deionized water was used, and further 300 μL of 25 mM chloroauric acid solution was added to the former solution.

### Example 16

A dispersion of core-shell nanoparticles having gold nanoshells was obtained in the same manner as in Example 8 except that in the above process of gold nanoshell formation on surface of core particles, no deionized water was used, and 100 μL of 75 mM chloroauric acid solution was added to the former solution.

### Example 17

A dispersion of core-shell nanoparticles having gold nanoshells was obtained in the same manner as in Example 8 except that in the above process of gold nanoshell formation on surface of core particles, the protective agent was changed into 5% by weight of polyvinyl alcohol (JP-05 manufactured by JAPAN VAM & POVAL CO., LTD., partially saponified type having an average polymerization degree of 500).

### Comparative Example 15

A dispersion of core-shell nanoparticles having gold nanoshells was obtained in the same manner as in Example 7 except that in the above process of gold nanoshell formation on surface of core particles, 325 μL of deionized water and 25 μL of 1 M bicine adjusted to pH 8 were used.

### Comparative Example 16

A dispersion of core-shell nanoparticles having gold nanoshells was obtained in the same manner as in Example 7 except that in the above process of gold nanoshell formation on surface of core particles, 300 μL of deionized water and 50 μL of 1 M bicine adjusted to pH 8 were used.

### Comparative Example 17

A dispersion of core-shell nanoparticles having gold nanoshells was obtained in the same manner as in Example 7 except that in the above process of gold nanoshell formation on surface of core particles, 250 μL of deionized water and 100 μL of 1 M bicine adjusted to pH 8 were used.

### Comparative Example 18

A dispersion of core-shell nanoparticles having gold nanoshells was obtained in the same manner as in Example 8 except that in the above process of gold nanoshell formation on surface of core particles, the pH of 1M bicine was changed to pH 7.4.

### Comparative Example 19

A dispersion of core-shell nanoparticles having gold nanoshells was obtained in the same manner as in Example 8 except that in the above process of gold nanoshell formation on surface of core particles, the protective agent was changed into 5% by weight of polyvinyl alcohol (JF-05 manufactured by JAPAN VAM & POVAL CO., LTD., completely saponified type having an average polymerization degree of 500).

### Comparative Example 20

A dispersion of core-shell nanoparticles having gold nanoshells was obtained in the same manner as in Example 8 except that in the above process of gold nanoshells formation on surface of core particles, the protective agent was changed into 5% by weight of polyvinyl alcohol (JF-10 manufactured by JAPAN VAM & POVAL CO., LTD., partially saponified type having an average polymerization degree of 1000).

### Test Example 1

The absorbance spectra of the core-shell nanoparticle dispersions having gold nanoshells obtained in Examples 1 and 2 and Comparative Examples 1 to 14 were measured with an absorptiometer (UV-1850, Shimadzu Corporation). The results are shown in Table 1. Based on the measured value of the maximum absorption wavelength and the calculated value of the maximum absorption wavelength according to the above section of Estimation of shell thickness, it was found that core-shell nanoparticles having gold nanoshells with a shell thickness of 9 nm to 11 nm were formed in Example 1, Example2, Comparative Example 2, and Comparative Example 14.

TABLE 1

| | | | | Concentration | Molar ratio | Type | Maximum |
|---|---|---|---|---|---|---|---|
| | Type of | Concentration | pH of | of chloroauric | between reducing | of | absorption |
| Example | protective | of protective | reducing | acid | agent and | reducing | wavelength |
| No. | agent | agent (%) | agent | (mM) | chloroauric acid | agent | (nm) |
| 1 | JP-10 | 0.42 | 8 | 6.3 | 20.0 | Reducing | 629.0 |
| | | | | | | agent1 | |
| 2 | JP-10 | 0.42 | 8 | 6.3 | 20.0 | Reducing | 650.0 |
| | | | | | | agent2 | |
| Comparative | Type of | Concentration of | pH of | Concentration of | Molar ratio between | Type of | Maximum |
| Example | protective | protective agent | reducing | chloroauric acid | reducing agent and | reducing | absorption |
| No. | agent | (%) | agent | (mM) | chloroauric acid | agent | wavelength (nm) |
| 1 | JP-10 | 0.42 | 8 | 6.3 | 20.0 | Reducing | 536.0 |
| | | | | | | agent3 | |
| 2 | JP-10 | 0.42 | 8 | 6.3 | 20.0 | Reducing | 637.0 |
| | | | | | | agent4 | |
| 3 | JP-10 | 0.42 | 8 | 6.3 | 20.0 | Reducing | 535.0 |
| | | | | | | agent5 | |
| 4 | JP-10 | 0.42 | 8 | 6.3 | 20.0 | Reducing | 726.0 |
| | | | | | | agent6 | |
| 5 | JP-10 | 0.42 | 8 | 6.3 | 20.0 | Reducing | 536.0 |
| | | | | | | agent7 | |
| 6 | JP-10 | 0.42 | 8 | 6.3 | 20.0 | Reducing | 670.0 |
| | | | | | | agent8 | |
| 7 | JP-10 | 0.42 | 8 | 6.3 | 20.0 | Reducing | 658.0 |
| | | | | | | agent9 | |
| 8 | JP-10 | 0.42 | 8 | 6.3 | 20.0 | Reducing | 529.0 |
| | | | | | | agent10 | |
| 9 | JP-10 | 0.42 | 8 | 6.3 | 20.0 | Reducing | 536.0 |
| | | | | | | agent11 | |
| 10 | JP-10 | 0.42 | 8 | 6.3 | 20.0 | Reducing | 751.0 |
| | | | | | | agent12 | |
| 11 | JP-10 | 0.42 | 8 | 6.3 | 20.0 | Reducing | 542.0 |
| | | | | | | agent13 | |
| 12 | JP-10 | 0.42 | 8 | 6.3 | 20.0 | Reducing | N.D. |
| | | | | | | agent14 | |
| 13 | JP-10 | 0.42 | 8 | 6.3 | 20.0 | Reducing | N.D. |
| | | | | | | agent15 | |
| 14 | JP-10 | 0.42 | 8 | 6.3 | 20.0 | Reducing | 633.0 |
| | | | | | | agent16 | |

The types of the protective agents in the table are as follows:

JP-10: polyvinyl alcohol, JP-10 manufactured by JAPAN VAM & POVAL CO., LTD., average polymerization degree 1000, and partially saponified type.

N.D. in the table means no data.

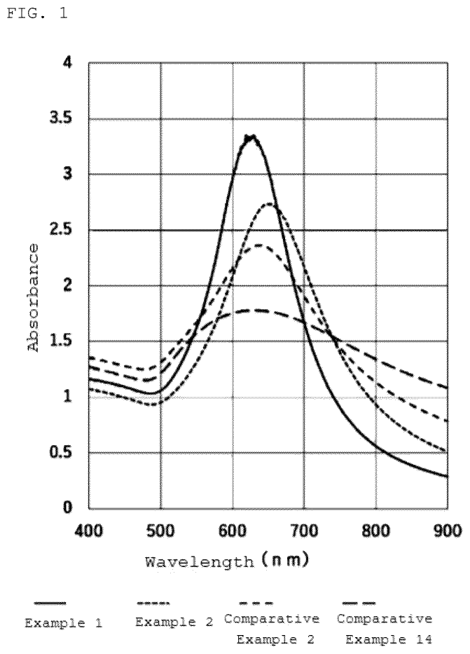

The absorbance spectra of the core-shell nanoparticle dispersions obtained in Examples 1 and 2, Comparative Example 2, and Comparative Example 14 are shown in FIG. 1.

As shown in FIG. 1, the absorbance spectra of the core-shell nanoparticle dispersions obtained in Examples 1 and 2 have a sharper peak as compared with those of Comparative Example 2 and Comparative Example 14. This indicates that the shell thickness of the core-shell nanoparticles of Examples 1 and 2 is more uniform than that of Comparative Example 2 and Comparative Example 14

### Test Example 2

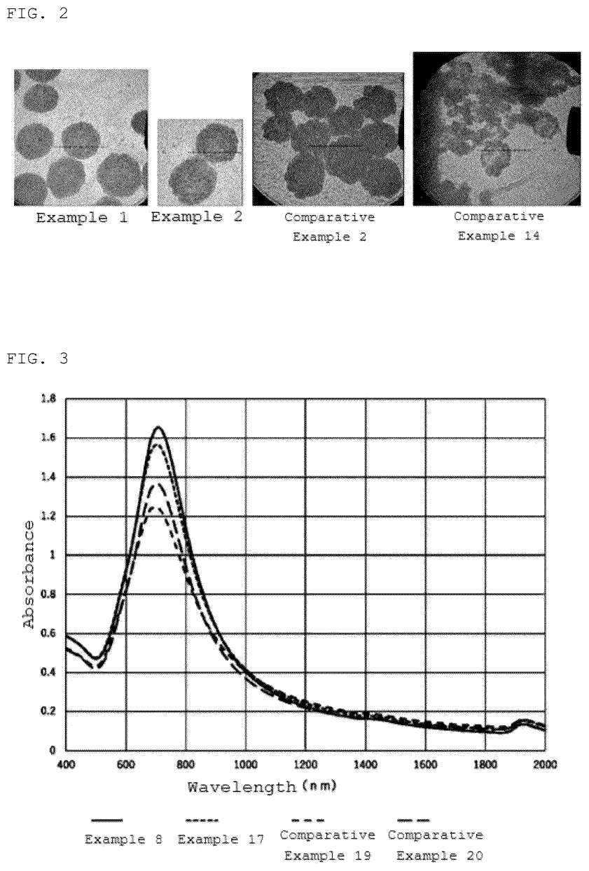

Transmission electron microscope images of the core-shell nanoparticles obtained in Examples 1 and 2, Comparative Example 2 and Comparative Example 14 are shown in FIG. 2. As a transmission electron microscope, JEM2010 manufactured by JEOL Ltd. was used.

It is found that the core-shell nanoparticles obtained in Examples 1 and 2 have denser gold nanoshells as compared with the core-shell nanoparticles obtained in Comparative Example 2 and Comparative Example 14.

### Test Example 3

The absorbance spectra of the core-shell nanoparticle dispersions having gold nanoshells obtained in Examples 3 to 17 and Comparative Examples 15 to 20 were measured with an absorptiometer (V-770, JASCO Corporation). The results are shown in Table 2.

TABLE 2

| | | Final | | Final | Molar ratio | Maximum |
|---|---|---|---|---|---|---|
| | Type of | concentration of | pH of | concentration | between reducing | absorption |
| Example | protective | protective agent | reducing | of chloroauric | agent and | wavelength |
| No. | agent | (%) | agent | acid (mM) | chloroauric acid | (nm) |
| 3 | DM-17 | 0.04 | 8 | 6.3 | 20.0 | 703.5 |
| 4 | DM-17 | 0.42 | 8 | 6.3 | 20.0 | 709.5 |
| 5 | DM-17 | 0.84 | 8 | 6.3 | 20.0 | 706.0 |
| 6 | DM-17 | 1.46 | 8 | 6.3 | 20.0 | 709.5 |
| 7 | ASP-05 | 0.84 | 8 | 6.3 | 16.7 | 709.0 |
| 8 | ASP-05 | 0.84 | 8 | 6.3 | 20.0 | 708.0 |
| 9 | ASP-05 | 0.84 | 8 | 6.3 | 26.7 | 705.0 |
| 10 | ASP-05 | 0.84 | 8 | 6.3 | 40.0 | 705.0 |
| 11 | ASP-05 | 0.84 | 7.7 | 6.3 | 20.0 | 704.0 |
| 12 | ASP-05 | 0.84 | 8.3 | 6.3 | 20.0 | 704.5 |
| 13 | ASP-05 | 0.84 | 8.6 | 6.3 | 20.0 | 690.5 |
| 14 | ASP-05 | 0.84 | 8 | 2.1 | 20.0 | 719.5 |
| 15 | ASP-05 | 0.84 | 8 | 10 | 20.0 | 696.5 |
| 16 | ASP-05 | 0.84 | 8 | 14 | 20.0 | 690.5 |
| 17 | JP-05 | 0.84 | 8 | 6.3 | 20.0 | 703.0 |
| | | Final | | | | Maximum |
| Comparative | Type of | concentration of | pH of | Concentration of | Molar ratio between | absorption |
| Example | protective | protective agent | reducing | chloroauric acid | reducing agent and | wavelength |
| No. | agent | (%) | agent | (mM) | chloroauric acid | (nm) |
| 15 | ASP-05 | 0.84 | 8 | 6.3 | 3.3 | 542.0 |
| 16 | ASP-05 | 0.84 | 8 | 6.3 | 6.7 | 556.5 |
| 17 | ASP-05 | 0.84 | 8 | 6.3 | 13.3 | 649.0 |
| 18 | ASP-05 | 0.84 | 7.4 | 6.3 | 20.0 | 634.5 |
| 19 | JF-05 | 0.84 | 8 | 6.3 | 20.0 | 699.0 |
| 20 | JP-10 | 0.84 | 8 | 6.3 | 20.0 | 702.5 |

The types of the protective agents in the table are as follows:
DM-17: carbonyl group-introduced modified polyvinyl alcohol (DM-17 manufactured by JAPAN VAM & POVAL CO., LTD., average polymerization degree 1700, and partially saponified type);
ASP-05: sulfonic acid group-introduced modified polyvinyl alcohol (ASP-05 manufactured by JAPAN VAM & POVAL CO., LTD., average polymerization degree 500, and partially saponified type);
JP-10: polyvinyl alcohol (JP-10 manufactured by JAPAN VAM & POVAL CO., LTD., average polymerization degree 1000, and partially saponified type);
JP-05: polyvinyl alcohol (JP-05 manufactured by JAPAN VAM & POVAL CO., LTD., average polymerization degree 500, and partially saponified type); and
JF-05: polyvinyl alcohol (JF-05 manufactured by JAPAN VAM & POVAL CO., LTD., average polymerization degree 500, and completely saponified type).

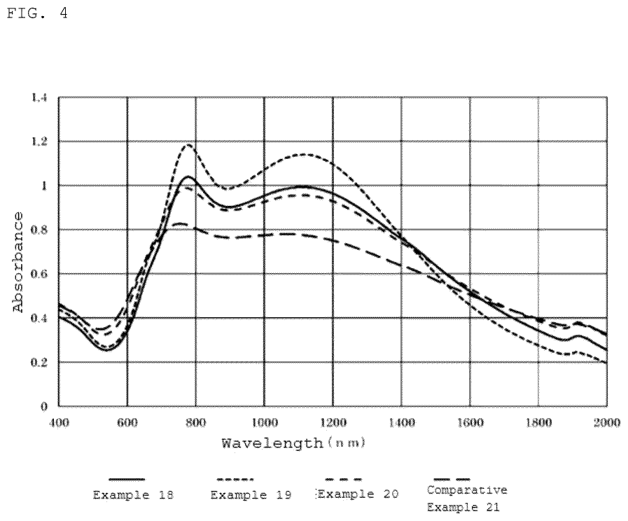

The absorbance spectra of the core-shell nanoparticle dispersions obtained in Example 8, Example 17, Comparative Example 19, and Comparative Example 20 are shown in FIG. 3. The absorbance at the maximum absorption wavelength was 1.65 in Example 8, 1.57 in Example 17, 1.25 in Comparative Example 19, and 1.37 in Comparative Example 20. As the dispersion stability of the obtained core-shell nanoparticles in an aqueous solution is higher, the recovery rate is higher, that is, lower recovery losses result in higher absorbance. In the production process using a high concentration of gold ion, which is a feature of the present invention, it is found that a partially saponified polyvinyl alcohol rather than a completely saponified polyvinyl alcohol (Example 17, Comparative Example 19), and a polyvinyl alcohol having an average polymerization degree of 500 rather than an average polymerization degree of 1000 (Example 17, Comparative Example 20) are suitable as a protective agent. The protective agent used in Example 8 is a partially saponified polyvinyl alcohol having an average polymerization degree of 500 and a sulfonic acid group-introduced modified polyvinyl alcohol. It is found that the core-shell nanoparticles are more highly dispersed and stabilized by the sulfonic acid group than normal polyvinyl alcohol.

### Example 18

### (Synthesis of SiO₂ Core Particles)

A 200 nm-diameter SiO₂ particle dispersion was obtained in the same manner as in Example 1 except that the reaction temperature was 4° C.

### (Introduction of Amino Group to Surface of SiO₂ Core Particles)

An amino group-introduced SiO₂ particle dispersion was obtained in the same manner as in Example 1 except that the SiO₂ particle dispersion having a particle diameter of 200 nm was used.

### (Synthesis of Seed Particles)

The same procedure as in Example 1 was performed to provide a seed particle dispersion.

(Formation of Gold Nanoshells on Surface of Core Particles) 220 μL of a seed particle dispersion and 100 μL of an amino group-introduced SiO₂ particle dispersion adjusted to 1% by weight were mixed in a 1.5 mL microtube, and the mixture was subjected to ultrasonic irradiation for 30seconds.

After the ultrasonic dispersion treatment, 100 μL of the solution was added to a 6 mL glass vial. To the glass vial, 200 μL of deionized water, 200 μL of 5% by weight of polyvinyl alcohol (JP-05 manufactured by JAPAN VAM & POVAL CO., LTD., average polymerization degree 500, partially saponified type), and 150 μL of 1 M bicine adjusted to pH 8 were added therein while stirring with a magnetic stirrer.

To the solution, 0.5 mL of a 15 mM chloroauric acid was added, and further the mixed solution was continuously stirred at 25° C. for 1 hour.

The reaction mixture was centrifuged at a centrifugal acceleration of 1,500 G for 5 minutes with a centrifuge to recover a precipitate, and a washing operation of redispersing the precipitate in 0.5 mL of a polyvinyl alcohol (JP-05 manufactured by JAPAN VAM & POVAL CO., LTD., partially saponified type having an average polymerization degree of 500) aqueous solution having a weight concentration of 0.1% was repeated 3 times. Finally, the mixture was dispersed in 0.5 mL of a 0.1% by weight of polyvinyl alcohol aqueous solution to provide a dispersion of core-shell nanoparticles having gold nanoshells. The absorbance of the obtained core-shell nanoparticle dispersion was measured.

### Example 19

A dispersion of core-shell nanoparticles having gold nanoshells was obtained in the same manner as in Example 18 except that in the above process of gold nanoshell formation on surface of core particles, 275 μL of deionized water, 75 μL of 1 M bicine adjusted to pH 8, and 0.5 mL of a mixed aqueous solution of 15 mM chloroauric acid and 15 mM potassium carbonate were added.

### Example 20

A dispersion of core-shell nanoparticles having gold nanoshells was obtained in the same manner as in Example 18 except that in the above process of gold nanoshell formation on surface of core particles, 300 μL of deionized water, 50 μL of 1 M bicine adjusted to pH 8, and 0.5 mL of a mixed aqueous solution of 15 mM chloroauric acid and 20 mM potassium carbonate were added.

### Comparative Example 21

A dispersion of core-shell nanoparticles having gold nanoshells was obtained in the same manner as in Example 18 except that in the above process of gold nanoshell formation on surface of core particles, 320 μL of deionized water, 30 μL of 1 M bicine adjusted to pH 8, and 0.5 mL of a mixed aqueous solution of 15 mM chloroauric acid and 24 mM potassium carbonate were added.

### Test Example 4

The absorbance spectra of the core-shell nanoparticle dispersions having gold nanoshells obtained in Examples 18 to 20 and Comparative Example 21 were measured with an absorptiometer (V-770, JASCO Corporation). The results are shown in Table 3. Based on the measured value of the maximum absorption wavelength and the calculated value of the maximum absorption wavelength according to the above section of Estimation of shell thickness, it was found that core-shell nanoparticles having gold nanoshells with a shell thickness of 9 nm to 11 nm were formed in Examples 18 to 20 and Comparative Example 21, but the maximum absorption wavelength of Comparative Example 21 was greatly shifted to a shorter wavelength side than the maximum absorption wavelength of Examples 18 to 20. In addition, it was found that the magnitude of the absorbance in Comparative Example 21 was lower than that in Examples 18 to 20.

TABLE 3

| | | Molar ratio between | | Maximum | |
|---|---|---|---|---|---|
| | Final concentration of | reducing agent and | Final concentration of | absorption | |
| Example No. | chloroauric acid (mM) | chloroauric acid | potassium carbonate (mM) | wavelength (nm) | Absorbance |
| 18 | 6.3 | 20 | 0 | 776.0 | 1.04 |
| 19 | 6.3 | 10 | 6.3 | 777.0 | 1.18 |
| 20 | 6.3 | 6.7 | 8.4 | 771.0 | 0.99 |
| | | Molar ratio between | | Maximum | |
| Comparative | Final concentration of | reducing agent and | Final concentration of | absorption | |
| Example No. | chloroauric acid (mM) | chloroauric acid | potassium carbonate (mM) | wavelength (nm) | Absorbance |
| 21 | 6.3 | 4 | 10.1 | 755.0 | 0.83 |

The absorbance spectra of the core-shell nanoparticle dispersions obtained in Example 18, Example 19, Example 20, and Comparative Example 21 are shown in FIG. 4. The absorbance at the maximum absorption wavelength was 1.04 in Example 18, 1.18 in Example 19, 0.99 in Example 20, and 0.83 in Comparative Example 21. In addition, the maximum absorption wavelength was 776 nm in Example 18, 777 nm in Example 19, 771 nm in Example 20, and 755 nm in Comparative Example 21. It was confirmed that chloroauric acid was neutralized by charging potassium carbonate, thus allowing to reduce the ratio of bicine serving as both a reducing agent and a pH buffer to chloroauric acid. When the ratio of chloroauric acid to bicine in Example 20 was less than 1:6.7, the maximum absorption wavelength and the absorbance significantly changed. As the dispersion stability of the obtained core-shell nanoparticles in an aqueous solution is higher, the recovery rate is higher, that is, lower recovery losses result in higher absorbance. It is found that the ratio of bicine to chloroauric acid is required to be 7 or more even when potassium carbonate is used as a neutralizing agent in the production process at a high concentration of gold ion, which is a feature of the present invention.

1. A method for producing core-shell particles having gold nanoshells and a protective agent on surfaces of core particles, comprising the following steps:
(a) mixing a solution of core particles and a solution of gold nanoclusters;
(b) adding a protective agent and a reducing agent and stirring, and further adding a gold complex to form gold nanoshells on surfaces of the core particles; and
(c) recovering the core-shell particles produced in the step (b), wherein
the reducing agent is a compound represented by Chemical Formula (1): 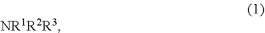
wherein R¹ is a C₁ to C₄ hydroxyalkyl group, R² is a C₁ to C₄ carboxyalkyl group, and R³ is a C₁ to C₄ hydroxyalkyl group, or a C₁ to C₄ carboxyalkyl group.

2. The method according to claim 1, wherein the reducing agent is bicine or N,N-bis(carboxymethyl)ethanolamine.

3. The method according to claim 1, wherein the reducing agent is bicine.

4. The method according to claim 1, wherein the protective agent is partially saponified polyvinyl alcohol having an average polymerization degree of 300 to 700.

5. The method according to claim 1, wherein the protective agent is modified partially saponified polyvinyl alcohol with an average polymerization degree of 300 to 700, having sulfonic acid group introduced. 6 (Original) The method according to claim 1, wherein in the step (b), the formation of gold nanoshells on surfaces of core particles is performed in the presence of a gold ion at a concentration of 5 mM or more.

7. The method according to claim 1, wherein in the step (b), the formation of gold nanoshells on surfaces of core particles is performed within 10 minutes at room temperature.

8. The method according to claim 1, wherein the core particles have a diameter of 50 nm to 300 nm.

9. The method according to claim 1, wherein the gold nanoshells have a thickness of 15 nm or less.

10. Core-shell nanoparticles comprising gold nanoshells and a protective agent on surfaces of core particles,
wherein the gold nanoshells have a thickness of 15 nm or less,
wherein the core particles have a diameter of 50 nm to 300 nm, and
wherein the protective agent is partially saponified polyvinyl alcohol with an average polymerization degree of 300 to 700
